(19) United States
(12) Patent Application Publication
(10) Pub. No.: US 2008/0188949 A1
(43) Pub. Date: Aug. 7, 2008

(54) Prosthetic and orthotic liners/sleeves with surfaces having a roughness or reduced coefficient of friction, and related methods

(76) Inventor: Craig MacKenzie, Orlando, FL (US)

Correspondence Address: Craig MacKenzie, 40 West Illiana Street, Orlando, FL, 32806, US

(21) Appl. No.: 12/011,579

(22) Filed: Jan. 28, 2008

Related U.S. Application Data
(60) Provisional application No. 60/898,857, filed on Feb. 1, 2007.

(51) Int. Cl. A61F 2/80

(52) U.S. Cl. 623/36

(57) ABSTRACT

A reliable prosthetic or orthotic skin interface liner includes surfaces having a roughness or reduced coefficient of friction. The liner is for use as a skin interface between a limb and a prosthetic or orthotic device. The liner includes an elastomeric material defining a tubular member having exterior and interior surfaces, and a matting agent added to the elastomeric material to reduce the coefficient of friction of at least one of the exterior and interior surfaces of the tubular member. After releasing the liner from a mold or press, exposing the liner to a gas, e.g. air, causes the surfaces of the liner to turn matte or slightly roughened. As such, the liner may be more easily applied to the patient's affected area.

### CROSS-REFERENCE TO RELATED APPLICATIONS

This application claims the benefit of U.S. provisional application No. 60/898,857 filed Feb. 1, 2007.

### BACKGROUND OF THE INVENTION

(1) Field of the Invention

The present invention relates to the field of prosthetic and orthotic liners and/or sleeves (i.e. skin-socket interface liners and sleeves), and more particularly to custom and production (“off the shelf”) prosthetic liners, sleeves, and associated methods.

(2) Discussion of Related Art

Liners provide a soft, flexible interface between a residual limb of an amputee and a hard socket to which a prosthetic device is secured. Such liners are typically made of an elastomer material such as silicone. Such liners may also be used in connection with orthotic devices. Suspension sleeves are a flexible tube used to secure the prosthetic device to the patients limb. The sleeve may be a sealing sleeve, or a suspension sleeve. Both types start on the prosthetic device and finish on the patients limb. Supportive sleeves can be used in an orthotic device to support a joint or limb of a patient.

Prosthetic suspension liners are described in prior patents, and may be fabricated of elastomer or rubber materials, and are used to cushion a post-operative stump or residual limb with respect to a prosthesis that is installed over the residual limb and coupled to the liner, e.g. by a conventional locking device.

Such liners should conform closely with the residual limb, accommodate all surface contours and sub-surface bone elements of the residual limb, and provide a comfortable cushion between the residual limb and the hard socket of the prosthesis that is to be fitted over the residual limb. Various silicone rubber or elastomer materials are used for suspension liners. Such elastomer materials having an appropriate hardness/softness, elongation, tensile, and other properties, such as bio-inertness (resulting in no skin reaction), have been successfully used for suspension liners.

Similar to liners, orthotic or prosthetic sleeves are provided for supporting and reinforcing muscles, joints, and extremities of patients, and also provide an airtight seal between a residual limb of an amputee and a prosthesis socket worn by the amputee. Moreover, such sleeves are not limited to use for amputees but may be applied to existing limbs to provide support in a manner associated with conventional orthotic devices. These sleeves may be similarly fabricated of elastomer materials. The sleeves may be cylindrical, curved or include other anatomically conforming shapes.

The elastomer forming the liner or sleeve frictionally engages and remains attached to the skin of a residual limb so that the limb is retained within the prosthetic socket in a comfortable, non-irritating manner. For example, liners may be used for any level of amputation both upper and lower limb.

Conventional approaches in liner and sleeve production have addressed the issue of tackiness in several different ways. The reason was to allow the patient to apply the liner or sleeve to the limb or affected area with ease. The tackiness of many conventional elastomer liners would not allow the liner to slide or invert on itself making it difficult for the patient to don the liner. Sleeves that have a tacky surface would sick to clothing causing unsightly bunching of the material.

Powder, e.g. baby powder, has been used to create a smooth non-tacky surface on the liner making it easier to apply, however, the baby powder had to be reapplied to the liner, e.g. every two to four days. This is a messy procedure and not one that a patient would readily do. Fabric covers have been used and were applied with glue. Such covers would often run peel or get dirty in the manufacturing process or through use by the amputee.

Coatings, such as Estane or other spray-on, paint or roll-on coatings, have been used to reduce the coefficient of friction of the surface of the liner but are not permanent. They flake, peel, or wear off after a period of time. Also, vapor deposition coatings, such as Parylene and other vapor deposition products, are applied to be permanent coatings. They require expensive machinery and a very delicate coating process. The coefficient of friction of the surface of the liner is reduced to allow for easy application of the liner to the patient, however, the coating is a higher durometer than the liner with less elasticity. When the liner is stretched the coating may crack, causing micro-tears in the surface of the liner. These micro-tears can harbor bacteria and be detrimental to the life of the liner.

### BRIEF SUMMARY OF THE INVENTION

It is an object of the present invention to provide a reliable prosthetic or orthotic skin interface liner with surfaces having a roughness or reduced coefficient of friction.

This and other objects, advantages and features in accordance with the present invention are provided by a liner for use as a skin interface between a limb and a prosthetic or orthotic device. The liner includes an elastomeric material defining a tubular member having exterior and interior surfaces, and a matting agent added to the elastomeric material to reduce the coefficient of friction of at least one of the exterior and interior surfaces of the tubular member.

The matting agent may reduce the coefficient of friction of both the exterior and interior surfaces of the tubular member. The elastomeric material may be a silicone or urethane material, for example. The matting agent preferably creates roughened exterior and interior surfaces of the tubular member. The tubular member may include an open end and a closed end.

Objects, advantages and features in accordance with the present invention are also provided by a method of making a liner for use as a skin interface between a limb and a prosthetic or orthotic device. The method includes: providing an elastomeric material; adding a matting agent to the elastomeric material to define a liner material; and forming a tubular member with the liner material and having exterior and interior surfaces. The matting agent added to the elastomeric material reduces the coefficient of friction of one or both of the exterior and interior surfaces of the tubular member.

Again, the elastomeric material may include a silicone or urethane material, for example. The matting agent preferably creates roughened exterior and interior surfaces of the tubular member. Furthermore, forming the tubular member may include forming an open end and a closed end, and/or may include pouring the liner material into a liner mold. Forming the tubular member may further include releasing the tubular member from the liner mold and exposing the exterior and interior surfaces of the tubular member to a gas, e.g. air.

### BRIEF DESCRIPTION OF THE SEVERAL VIEWS OF THE DRAWING(S)

FIG. 1 is an elevational view of a liner in accordance with features of the present invention.

FIG. 2 is a schematic diagram of an embodiment of a liner manufacturing technique for making the liner of FIG. 1.

FIG. 3 is a flowchart illustrating the various steps of a method of making the liner of FIG. 1.

### DETAILED DESCRIPTION OF THE PREFERRED EMBODIMENTS

The present invention is directed to a prosthetic or orthotic liner and associated methods of making and using, and includes the use of an elastomer material, e.g. pourable or injectable silicone, that may be used with a simple mold or press. The silicone is preferably biocompatible, e.g. “healthcare grade” or “medical grade”, which is suitable for external use. For example, an appropriate silicone system may also be clear to semi-translucent and curable at room temperature. The molded silicone liner should have high tear strength and exhibit flexibility and high elasticity.

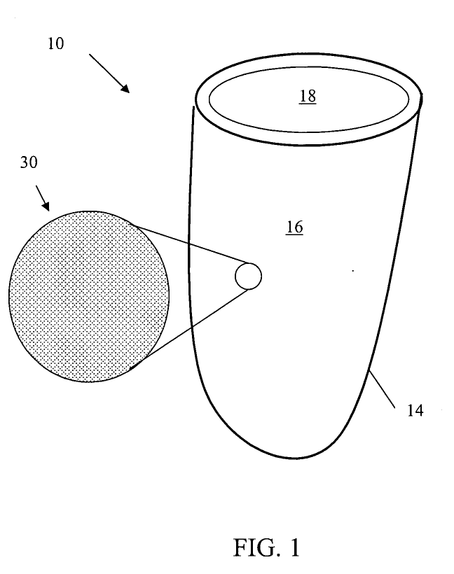

Referring initially to FIGS. 1-3, a reliable prosthetic or orthotic skin interface liner 10 with surfaces having a roughness or reduced coefficient of friction will now be described. The liner 10 is for use as a skin interface between a limb and a prosthetic or orthotic device, as would be appreciated by those skilled in the art, and as discussed above. The liner 10 includes an elastomeric material 12 defining a tubular member 14 having exterior and interior surfaces 16, 18. A matting agent 20 is added to the elastomeric material 12 to reduce the coefficient of friction of one or both of the exterior and interior surfaces 16, 18 of the tubular member 14.

As illustrated, the tubular member 14 may include an open end 22 and a closed end 24. The matting agent 20 preferably creates roughened exterior and interior surfaces 16, 18 of the tubular member 14, e.g. as illustrated in the enlarged portion 30 of FIG. 1. The reduced coefficient of friction of the exterior and interior surfaces 16, 18 of the liner 10 allows the liner to slide or invert on itself making it easier for the patient to don the liner.

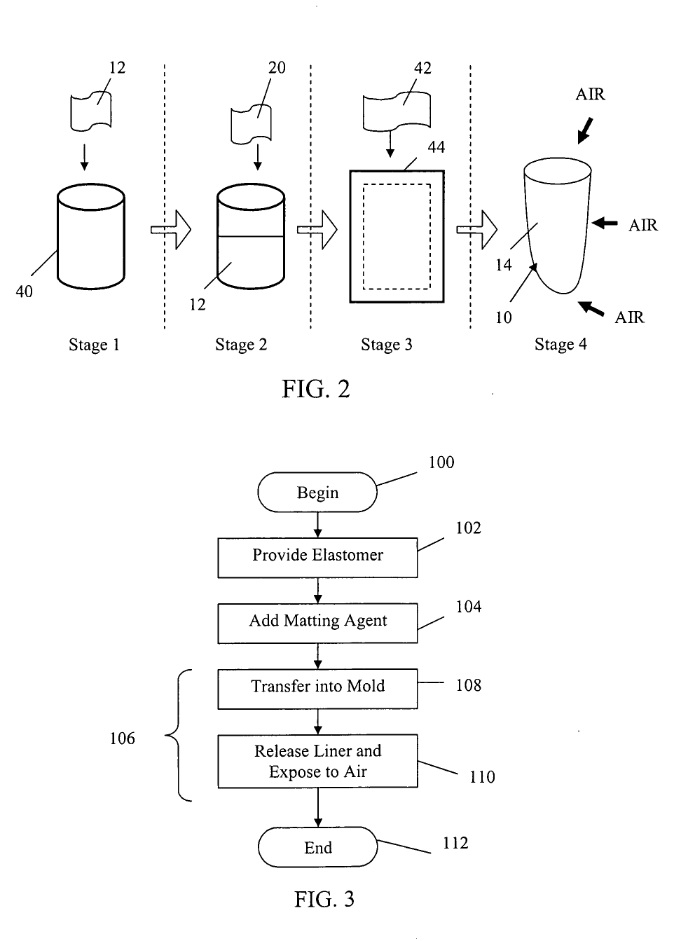

Referring more specifically to FIGS. 2 and 3, a method of making the liner 10 will now be described. Illustratively, FIG. 2 includes Stages 1-4, and FIG. 3 includes steps 100-112, of an embodiment of the liner manufacturing process. The method begins (step 100) and at Stage 1 (step 102), the elastomeric material 12, such as silicone or urethane, is provided, e.g. in a container 40. At Stage 2 (step 104), the matting agent 20 is added to the elastomeric material 12 to define a liner material 42.

At Stages 3 and 4, corresponding to step 106, the method includes forming the tubular member 14 with the liner material 42. Forming the tubular member 14 may include forming an open end and a closed end as discussed above. The matting agent 20 added to the elastomeric material 12 reduces the coefficient of friction of one or both of the exterior and interior surfaces of the tubular member 14. The matting agent 20 preferably creates roughened exterior and interior surfaces of the tubular member. Step 106 illustratively includes, for example, pouring the liner material 42 into a liner mold 44 (step 108), and then subsequently (at step 110) releasing the tubular member 14 from the liner mold 44 and exposing the exterior and interior surfaces of the tubular member to a gas, e.g. air, before ending the method at step 112.

The matting agent 20 or additive is used to create a matted surface or micro-roughness of the liner surfaces 16, 18. Although the description refers to silicone liners, such a matting agent or additive could be used for urethane, styrene, or other elastomers for liners. In a preferred embodiment, the matting agent 20 is added to silicone during mixing either by hand or by metered mixing device. After the liner 10 is released from the mold 44, the additive causes the surface of the liner 10 to turn matte or slightly roughened after a certain period of exposure to a gas, e.g. air. The matting agent reduces the coefficient of friction of the external and internal surface of the liner 10 enough so that the liner may be more easily applied to the patient's affected area.

An example of a preferable matting agent is manufactured by Quantum Silicones, Inc. 8021 Reycan Rd. Richmond, Va. 23237 http://www.quantumsilicones.com. In an embodiment, the matting agent is mixed with the silicone at 0.001% to 10% of the total weight of the silicone with the preferred percentage being 0.065%.

The present invention was described with references to preferred embodiments of the invention. This invention may, however, be embodied in many different forms and should not be construed as limited to the specific embodiments set forth herein. Rather, these embodiments are provided so that this disclosure will be thorough and complete, and will fully convey the scope of the invention to those skilled in the art. Many modifications and other embodiments of the invention will come to the mind of one skilled in the art having the benefit of the teachings presented in the foregoing descriptions and the associated drawings.

1. A liner for use as a skin interface between a limb and a prosthetic or orthotic device, the liner comprising:
an elastomeric material defining a tubular member having exterior and interior surfaces; and
a matting agent added to the elastomeric material to reduce the coefficient of friction of at least one of the exterior and interior surfaces of the tubular member.

2. The liner according to claim 1 wherein the matting agent reduces the coefficient of friction of both the exterior and interior surfaces of the tubular member.

3. The liner according to claim 1 wherein the elastomeric material comprises a silicone material.

4. The liner according to claim 1 wherein the elastomeric material comprises a urethane material

5. The liner according to claim 1 wherein the matting agent creates roughened exterior and interior surfaces of the tubular member.

6. The liner according to claim 1 wherein the tubular member includes an open end and a closed end.

7. A liner for use as a skin interface between a limb and a prosthetic or orthotic device, the liner comprising:
an elastomeric material defining a tubular member having exterior and interior surfaces; and
a matting agent in the elastomeric material to create roughened exterior and interior surfaces of the tubular member.

8. The liner according to claim 7 wherein the matting agent reduces the coefficient of friction of both the exterior and interior surfaces of the tubular member.

9. The liner according to claim 7 wherein the elastomeric material comprises a silicone material.

10. The liner according to claim 7 wherein the elastomeric material comprises a urethane material

11. The liner according to claim 1 wherein the tubular member includes an open end and a closed end.

12. A method of making a liner for use as a skin interface between a limb and a prosthetic or orthotic device, the method comprising:
providing an elastomeric material;
adding a matting agent to the elastomeric material to define a liner material; and
forming a tubular member with the liner material and having exterior and interior surfaces;
the matting agent added to the elastomeric material reducing the coefficient of friction of at least one of the exterior and interior surfaces of the tubular member.

13. The method according to claim 12 wherein the matting agent reduces the coefficient of friction of both the exterior and interior surfaces of the tubular member.

14. The method according to claim 12 wherein the elastomeric material comprises a silicone material.

15. The method according to claim 12 wherein the elastomeric material comprises a urethane material

16. The method according to claim 12 wherein the matting agent creates roughened exterior and interior surfaces of the tubular member.

17. The method according to claim 12 wherein forming the tubular member includes forming an open end and a closed end.

18. The method according to claim 12 wherein forming the tubular member includes pouring the liner material into a liner mold.

19. The method according to claim 18 wherein forming the tubular member further includes releasing the tubular member from the liner mold and exposing the exterior and interior surfaces of the tubular member to a gas.

20. The method according to claim 19 wherein the gas comprises air.
